(19) United States
(12) Patent Application Publication
(10) Pub. No.: US 2018/0169547 A1
(43) Pub. Date: Jun. 21, 2018

(54) APPARATUS FOR MICROCARRIER FILTRATION AND SEPARATION FROM CELLS AND MEDIA

(71) Applicant: Corning Incorporated, Corning, NY (US)

(72) Inventor: William Joseph Lacey, North Andover, MA (US)

(21) Appl. No.: 15/579,786

(22) PCT Filed: Jun. 8, 2016
(86) PCT No.: PCT/US16/36282

§ 371 Date: Dec. 5, 2017

Related U.S. Application Data
(60) Provisional application No. 62/172,454, filed on Jun. 8, 2015.

(51) Int. Cl. B01D 29/58; C12M 1/00; C12M 1/12; C12M 1/26; C12N 1/02

(52) U.S. Cl. CPC B01D 29/58; C12M 47/02; C12N 1/02; C12M 25/16; C12M 33/14; C12M 1/126

(57) ABSTRACT

A flow-through apparatus is provided. The flow-through apparatus includes a housing having a fixed volume and having an inlet, an outlet, and a cavity. The flow-through apparatus also includes a first filter in fluid communication with the inlet and disposed within the cavity and a seal attaching the first filter to the housing. The first filter has a mesh size of about 100 microns or less and a first internal volume less than the fixed volume of the housing.

This application claims the benefit of priority under 35 U.S.C. § 119 of U.S. Provisional Application Ser. No. 62/172,454 filed on Jun. 8, 2015, the content of which is relied upon and incorporated herein by reference in its entirety.

### FIELD OF THE DISCLOSURE

The present disclosure relates generally to apparatuses and methods for filtering microcarriers, and, more particularly, to flexible and rigid flow-through filters for separating microbeads from cells and growth media.

### BACKGROUND

Cell culture can be an important step in many human and animal treatments and therapies, for example, stem cell therapy and/or vaccine production, to name a few applications. Recent developments in cell culture have shown that it is possible to culture cells on microcarriers. For example, microbeads can be used to grow adhesion/adherent type cells in flasks or bioreactors. Depending on the particular process, the microbeads can be separated from growth media and cells either before or after cell trypsinization and common separation methods may include the use of in-line filters and open sieves. However, current methods for separating microbeads have several disadvantages, e.g., process blockage or bottlenecking, cell contamination, and/or material loss.

When utilizing an in-line filter, e.g., a mesh placed over a fitting, port, tube, or other opening along the process line, such mesh filters often capture a significant quantity of cells along with the microbeads. Additionally, these filters tend to clog easily, thereby backing up or bottlenecking the process flow until the obstruction is cleared. Furthermore, current in-line filter construction does not allow for the easy recovery of the separated microbeads.

Open sieves can also be used to separate microbeads, for example, by pouring a mixture including microbeads, growth media, and cells from a flask or other container through the sieve. However, pouring such media through an open sieve is not ideal as this technique often leads to cell contamination and/or material loss or spillage.

Accordingly, it would be advantageous to provide methods and apparatuses which address the above drawbacks, e.g., a filter that efficiently separates microbeads and/or cells and growth media and allows for the easy recovery of the separated microbeads and/or cells.

### SUMMARY

The disclosure relates, in various embodiments, to flow-through apparatuses for separating microbeads and/or cells from growth media. According to an embodiment, the apparatus includes a housing having a fixed volume and including an inlet, an outlet, and a cavity; a first filter in fluid communication with the inlet and disposed within the cavity; and a seal attaching the first filter to the housing; wherein the first filter has a mesh size of about 100 microns or less and a first internal volume less than the fixed volume of the housing. The first filter may be concentrically disposed within the cavity.

According to an embodiment, the flow-through apparatus can include a second filter, wherein the first filter is concentrically disposed within the second filter and the second filter has a mesh size of about 5 microns or less and a second internal volume greater than the first internal volume of the first filter and less than the fixed volume of the housing.

According to an embodiment, the apparatus is a flexible bag including first and second sidewalls sealed along a periphery to define a cavity including an inlet and an outlet; and a filter disposed within the cavity and having a mesh size of about 100 microns or less; wherein the filter is attached to (a) an inside surface of the first sidewall at a first interface located along a length of the bag and (b) an inside surface of the second sidewall at a second interface located along the length of the bag; and wherein the length of the bag extends from the inlet to the outlet and the first interface is nearer to the inlet than the second interface.

Further disclosed herein are methods for filtering a medium (e.g., a medium including microbeads and/or cells). According to an embodiment, the method includes introducing a medium into a flow-through apparatus including a housing having a fixed volume and including an inlet, an outlet, and a cavity; a first filter in fluid communication with the inlet and (concentrically) disposed within the cavity, wherein the first filter has a mesh size of about 100 microns or less and a first internal volume less than the fixed volume of the housing; and a seal attaching the first filter to the housing; wherein the medium enters the housing inlet and flows into the housing cavity through the first filter to produce a filtrate and the filtrate exits the housing cavity through the outlet.

According to an embodiment, the method includes introducing a medium into a flow-through apparatus including a flexible bag including first and second sidewalls sealed along a periphery to define a cavity including an inlet and an outlet; and a filter disposed within the cavity and having a mesh size of about 100 microns or less; wherein the filter is attached to (a) an inside surface of the first sidewall at a first interface located along a length of the bag and (b) an inside surface of the second sidewall at a second interface located along the length of the bag; wherein the length of the bag extends from the inlet to the outlet and the first interface is nearer to the inlet than the second interface; and wherein the medium enters the bag inlet and flows through the first filter to produce a filtrate and the filtrate exits through the outlet.

Additional features and advantages of the present disclosure will be set forth in the detailed description which follows, and in part will be readily apparent to those skilled in the art from that description or recognized by practicing the embodiments as described herein, including the detailed description which follows, the claims, and the appended drawings.

It is to be understood that both the foregoing general description and the following detailed description present various embodiments of the disclosure, and are intended to provide an overview or framework for understanding the nature and character of the claims. The accompanying drawings are included to provide a further understanding of the disclosure, and are incorporated into and constitute a part of this specification. The drawings illustrate various embodiments of the disclosure and together with the description serve to explain the principles and operations of the embodiments of the present disclosure.

### BRIEF DESCRIPTION OF THE DRAWINGS

The following detailed description can be best understood when read in conjunction with the following drawings, in which, where possible, like numerals are used to refer to like elements, and:

FIG. 1 is a schematic illustrating an embodiment of a flow-through apparatus according to certain embodiments of the disclosure;

FIG. 2 is a cross-sectional view of an inner filter housing for a flow-through apparatus according to various embodiments of the disclosure;

FIG. 3 is a cross-sectional view of the flow-through apparatus depicted in FIG. 1;

FIG. 4 is a cross-sectional view of a flow-through apparatus having first and second filters according to certain embodiments of the disclosure;

FIG. 5 is a schematic illustrating a flow-through apparatus according to certain embodiments of the disclosure; and

FIG. 6 is a cross-sectional view of the flow-through apparatus depicted in FIG. 5.

### DETAILED DESCRIPTION

Apparatuses

Disclosed herein is a flow-through apparatus for the filtering and separation of microbeads from cells and growth media, an embodiment of the apparatus including a housing having a fixed volume and including an inlet, an outlet, and a cavity; a first filter in fluid communication with the inlet and disposed, e.g., concentrically, within the cavity; and a seal attaching the first filter to the housing; wherein the first filter has a mesh size of about 100 microns or less and a first internal volume less than the fixed volume of the housing.

According to an embodiment, the flow-through apparatus for filtering and separation of microbeads from cells and growth media includes a flexible bag including first and second sidewalls sealed along a periphery to define a cavity including an inlet and an outlet; and a filter disposed within the cavity and having a mesh size of about 100 microns or less; wherein the filter is attached to (a) an inside surface of the first sidewall at a first interface located along a length of the bag and (b) an inside surface of the second sidewall at a second interface located along the length of the bag; and wherein the length of the bag extends from the inlet to the outlet and the first interface is nearer to the inlet than the second interface.

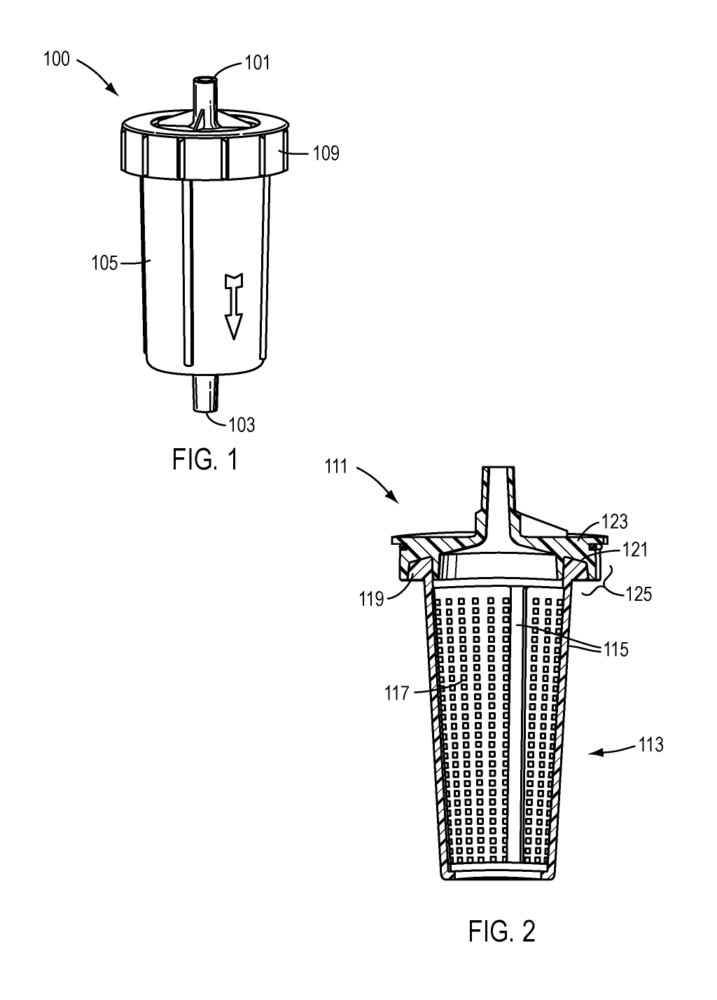

Various embodiments of the disclosure will be discussed with reference to FIGS. 1-7, which illustrate schematics of flow-through apparatuses for filtering and separation of microbeads from cells and growth media according to non-limiting embodiments of the disclosure. The following general description is intended to provide an overview of the claimed apparatuses and various aspects will be more specifically discussed throughout the disclosure with reference to the non-limiting embodiments, these embodiments being interchangeable with one another within the context of the disclosure.

The terms “microcarriers,” “microbeads,” and “beads” are used interchangeably herein to refer to cell culture articles. Microcarriers (e.g., microbeads) may be rounded and/or spherical in shape, but are not constrained by such a shape and may have any other shape, including regular and irregular shapes, suitable for culturing cells on one or more surfaces of the microcarrier.

Example cell culture articles are microcarriers, which are also referred to as beads or microbeads (collectively “microcarriers”).”

FIG. 1 illustrates an external view of an embodiment of a flow-through apparatus 100 for filtering and separation of microbeads from cells and growth media. The flow-through apparatus 100 includes a filter housing 105 having an inlet 101, an outlet 103, and a cavity (not illustrated). The filter housing 105 can also be equipped with a top 109 for reversibly opening and closing the filter housing 105. The housing 105 and the top 109 can, in some embodiments, be constructed of a substantially rigid material, such as polypropylene or polycarbonate, and the like. The top 109 and housing 105 can further be equipped with a closing mechanism, such as mated threading, e.g., a threaded flange on the interior of the top 109 can couple to a threaded flange on the exterior of the housing 105.

A first filter can be positioned in the cavity of the filter housing 105. The first filter 113 will be discussed in part with reference to FIG. 2, which depicts a cross-sectional view of an inner filter housing 111. In some embodiments, as illustrated, the first filter 113 can includes a filter frame 115 and a mesh 117. The filter frame 115 can be constructed from a substantially rigid material, such as polypropylene or polycarbonate, and like materials. The mesh 117 can be constructed from a substantially flexible or substantially rigid material, such as polypropylene, polyester, or polycarbonate.

In some embodiments, the first filter 113 can include a filter frame 115 and separate mesh 117 (either rigid or flexible) which can be a removed from the filter frame 115 for cleaning/replacement. Alternatively (not illustrated) the first filter 113 can be a one-piece molded filter which can be removed from the housing 105 for cleaning/replacement. The first filter 113 can include one material or two or more materials. For instance, the first filter 113 can be a single piece molded from a single material; a single piece having two or more materials, e.g., constructed by overmolding one material over another or otherwise attaching two or more materials; or a multi-component piece having two or more pieces made from the same or different materials.

The first filter 113 can have an internal volume, which can be defined by the filter frame 115 and/or mesh 117. In certain embodiments, the first filter 113 can have a substantially cylindrical shape (as illustrated), although other shapes, such as conical or frusto-conical shapes, are possible and envisioned as falling within the scope of the disclosure. The mesh size of the first filter 113 can be about 100 microns or less, such as from about 10 microns to about 80 microns, from about 20 microns to about 70 microns, from about 30 microns to about 60 microns, or from about 40 microns to about 50 microns, including all ranges and subranges therebetween.

According to certain embodiments, the first filter 113 can include an extending feature 119 configured to interlock with a recessed feature 121 of an inner filter housing cap 123. The extending feature 119 can be located proximate the inlet or at an “upstream” location on the first filter 113. As used herein the term “upstream” is intended to denote that the referenced component is closer to the flow-through apparatus inlet 101 than its outlet 103. In various embodiments, the inner filter housing cap 123 can be removably secured to the first filter 113 by a “snap-fit” or other mating mechanism in which the extending feature 119 of the first filter 113 can be mated with a corresponding recessed feature 121 of the inner filter housing cap 123. The mated features 119 and 121 can, together, make up an inner filter housing protrusion 125.

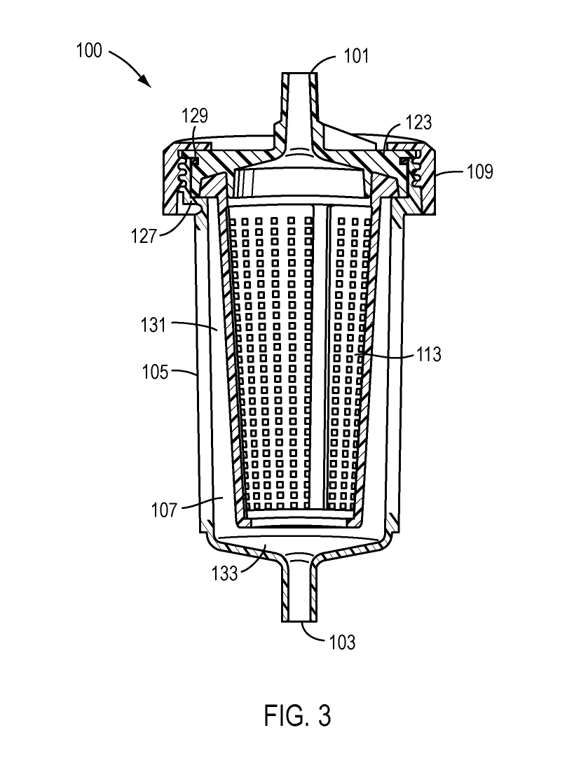

Referring to the cross-sectional view of the apparatus 100 in FIG. 3, the inner filter housing 111 (not labeled) can be positioned within the filter housing 105 such that the first filter 113 is concentrically disposed within the inner cavity 107. For example, the protrusion 125 in FIG. 2 (not labeled in FIG. 3) can, in some embodiments, abut or rest on a lip 127 located on an upstream portion of an internal surface of the filter housing 105. In some embodiments, the inner filter housing 111 (not labeled) can be secured between the lip 127 and the top 109 of the filter housing 105 when in the closed position. A seal 129, such as an O-ring seal, on the outer periphery of the inner filter housing cap 123 can provide a seal between the filter housing 105 and the inner filter housing cap 123. The first filter 113 can thus be reversibly secured, attached, or sealed to the filter housing 105.

According to various embodiments, the filter housing 105 has a fixed volume and the first filter 113, concentrically disposed in the cavity 107 of the filter housing 105, has a volume less than the fixed volume of the filter housing 105. In the illustrated configuration, the first filter 113 is positioned to provide a peripheral gap 131 and basal gap 133 between the inner walls of the filter housing 105 and the external surface of first filter 113. The internal volume of the first filter can, in some embodiments, correspond to the following formula: V_{filter1}=X*V_{housing}, where X can range from about 0.1 to about 0.95, such as from about 0.2 to about 0.9, from about 0.3 to about 0.8, from about 0.4 to about 0.7, or from about 0.5 to about 0.6, including all ranges and subranges therebetween. The “free” volume of the apparatus, e.g., the combined volume of the basal and peripheral gap regions not overlapping with the internal volume of the first filter can thus be defined as V_{free}=V_{housing}−V_{filter1}. The ratio of the first filter volume to the free volume can range, for instance, from about 95:5 to about 10:90, such as from about 90:10 to about 20:80, from about 80:20 to about 30:70, from about 70:30 to about 40:60, or from about 60:40 to about 50:50, including all ranges and subranges therebetween. The values for each of these volumes can vary as desired for a particular application and it is within the ability of one skilled in the art to choose a suitable filter configuration.

In additional embodiments, the flow-through apparatus can include two filters, e.g., two concentric filters. The configuration may be similar to the embodiment depicted in FIG. 3 utilizing a single filter. As described above, the first filter mesh size can be about 100 microns or less, such as from about 10 microns to about 80 microns, from about 20 microns to about 70 microns, from about 30 microns to about 60 microns, or from about 40 microns to about 50 microns, including all ranges and subranges therebetween. The second filter mesh size can, in some embodiments, be about 5 microns or less, such as about 4 microns or less, about 3 microns or less, about 2 microns or less, or about 1 micron or less, including all ranges and subranges therebetween.

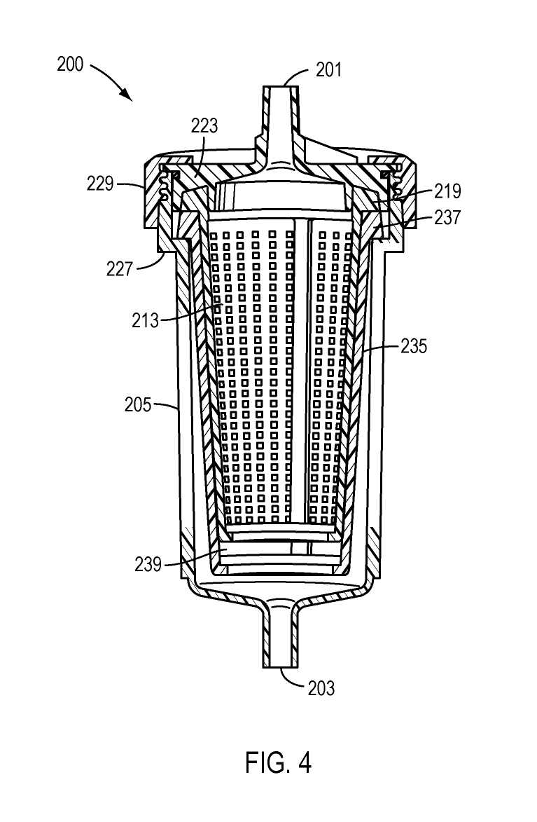

With reference to FIG. 4, a second filter 235, having a volume greater than the first filter volume but less than the housing filter volume can be positioned in the housing cavity 207 of apparatus 200, e.g., the second filter 235 can be positioned inside the housing cavity 207 and the first filter 213 can be positioned inside the second filter 235. For instance, the first filter 213 can be concentrically disposed in the second filter 235, wherein an intermediate basal gap 239 is provided between the outer surface of the base of the first filter 213 and the inner surface of the base of the second filter 235. Although not readily perceived in the illustration provided in FIG. 4, an intermediate peripheral gap (not labeled) can also be present between the sidewalls of the first and second filters 213, 235. Similar to the embodiment utilizing a single filter, peripheral and basal gaps 231, 233 may also be provided between the second filter 235 and the interior surfaces of the filter housing 205.

The second filter 235 can be substantially similar in construction to the first filter 213 in terms of rigidity, shape, and/or materials, these materials and shapes being chosen from those listed above with reference to the first filter 213. However, it is also possible to use first and second filters having different shapes, sizes, and or materials. According to various embodiments, the second filter 235 can include a second extending feature 237 similar to the first extending feature 219 of the first filter.

As concentrically assembled in FIG. 4, the first filter extending feature 219 may abut or be positioned on or proximate the second filter extending feature 237. The first filter extending feature 219 and second filter extending feature 237 can be utilized to removably secure the first filter 213 and second filter 235 to the inner filter housing cap 223 using the “snap-fit” or other mating mechanism described above, wherein the first filter extending feature 219 is sandwiched between the second filter extending feature 237 and the recessed portion 221 of the inner filter housing cap 223. In this embodiment, the inner filter housing (not illustrated) can include the inner filter housing cap 223, the first filter 213, and the second filter 235. The second filter extending feature 237 and the inner filter housing cap 223 can be positioned on the lip 227 in the upstream portion of the internal surface of the filter housing 205, thereby securing the inner filter housing cap 223, the first filter extending feature 219, and the second filter extending feature 237 between the lip 227 and the top 209. The first filter 213 and second filter 235 can thus be reversibly secured, attached, or sealed to the filter housing 205.

In the apparatus 200 illustrated in FIG. 4 having two concentric filters, the volume of the second filter 235 can be calculated as: V_{filter2}=X*V_{housing}, where X ranges from about 0.1 to about 0.95, such as from about 0.2 to about 0.9, from about 0.3 to about 0.8, from about 0.4 to about 0.7, or from about 0.5 to about 0.6, including all ranges and subranges therebetween. Similarly, the volume of the first filter 213 can be calculated as: V_{filter1}=X*V_{filter2}, where X ranges from about 0.1 to about 0.95, such as from about 0.2 to about 0.9, from about 0.3 to about 0.8, from about 0.4 to about 0.7, or from about 0.5 to about 0.6, including all ranges and subranges therebetween. Finally, the “free” volume of the housing 205 can be calculated as: V_{free}=V_{housing}−V_{filter2}. The “intermediate” volume in the housing, e.g., the volume bounded by the second filter but not bounded by the first filter (second basal gap+second peripheral gap volume), can be calculated as: V_{intermediate}=V_{filter2}−V_{filter1}. The values for each of these volumes can vary as desired for a particular application and it is within the ability of one skilled in the art to choose a suitable filter configuration.

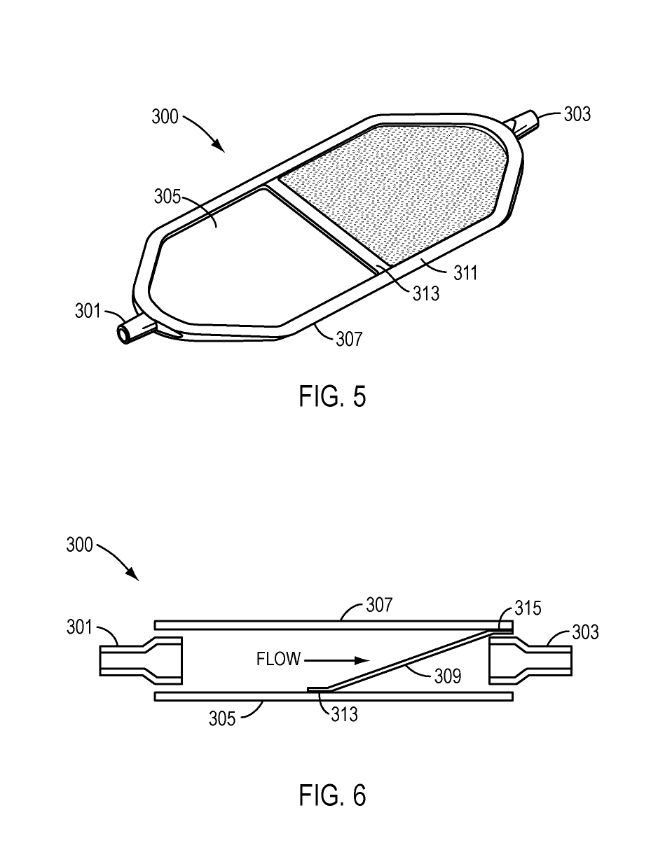

FIG. 5 illustrates an external view of an additional embodiment of a flow-through apparatus (or flexible bag) 300 for filtering and separation of microbeads from cells and growth media. The flow-through apparatus includes an inlet 301, an outlet 303, a first sidewall 305, a second sidewall 307, and a filter 309. The first and second sidewalls 305, 307 are sealed along a periphery 311 to form a cavity (not labeled). In the illustrated embodiment, the first and second sidewalls 305, 307 are illustrated as transparent (thus the filter 309 inside the bag is visible via the external view). Of course, the first and/or second sidewalls 305, 307 need not be transparent and can be opaque and/or colored if desired. The filter 309 can be attached to the first sidewall 305 at a first interface 313 along the length of the bag. Similarly, a second interface (not visible) can connect the filter 309 to the second sidewall 307. According to various embodiments, and as illustrated, one of the first or second interfaces can be at least partially coextensive with the peripheral seal 311 proximate the inlet or outlet.

FIG. 6 provides a cross-sectional view of the apparatus (or flexible bag) 300 in which the second interface 315 is visible. In the non-limiting embodiments illustrated in FIGS. 5-6, the first interface 313 is positioned approximately mid-way along the length of the bag and the second interface is positioned proximate the outlet. However, this configuration is not limiting and any other interface configuration is possible and envisioned. For example, the first interface could be positioned at the inlet end and the second interface can be positioned along the length of the bag (e.g., approximately at the midway point). In other embodiments, the first and/or second interfaces can be positioned at any point along the length of the bag, e.g., for a bag length L, the first interface can be positioned at a location Y*L, where Y is equal to 0, 0.1, 0.2, 0.3, 0.4, 0.5, 0.6, 0.7, 0.8, or 0.9 (Y=0 representing the inlet), including all ranges, and subranges therebetween. Similarly, the second interface can be positioned at a location Z*L, wherein Z is equal to 0.1, 0.2, 0.3, 0.4, 0.5, 0.6, 0.7, 0.8, 0.9 or 1 (Z=1 representing the outlet), including all ranges, and subranges therebetween. In some embodiments, the first interface can be located proximate the inlet (e.g., at or near 0*L) and the second interface can be located proximate the outlet (e.g., at or near 1*L). According to additional embodiments, the first and/or second interfaces can be located in a central region along the length of the bag (e.g., at or near 0.5*L).

Any combination of these positions is envisioned and possible, so long as the first interface is closer to the inlet than the second interface (e.g., such that the first and second interface are not located at the same position along the length of the bag). In some embodiments, the filter 309 is disposed in the bag such that it does not form a 90° angle with either the first or second sidewall. According to various embodiments, the filter 309 forms an angle with the first or second sidewall ranging from about 1° to about 89°, such as from about 10° to about 80°, from about 20° to about 70°, from about 30° to about 60°, or from about 40° to about 50°, including all ranges and subranges therebetween. Without wishing to be bound by theory, it is believed that positioning the filter 309 in the bag 300 at an angle skewed from 90° can provide additional filter surface area through which the medium can flow, thereby possibly reducing clogging and/or bottlenecking in the filter bag.

The first sidewall 305 and second sidewall 307 can be constructed from any suitable flexible material, e.g., plastics such as polypropylene or polyethylene. The peripheral seal 311 between first and second sidewalls 305, 307 can be formed in any manner known in the art, e.g., using a heat seal or adhesives, to name a few. The inlet 301 and outlet 303 can, in some embodiments, include barbs, fittings, tubes, ports, or the like, and can be formed and attached to the flexible bag 300 by any suitable method. For example, the inlet 301 and outlet 303 can include barbs formed by injection molding at opposite ends of the filter bag. According to non-limiting embodiments, the inlet 301 and outlet 303 can be separate pieces (e.g., barbs) attached to the bag 300, e.g., by heat or impulse sealing. In the embodiments illustrated in FIGS. 5-6, the inlet and outlet may be positioned and configured to allow for flow through the bag along the axis of the peripheral seal 311. The material of construction of the inlet 301 and outlet 303 can vary depending on the material of construction of the first and second sidewall liners 305, 307, and can include propylene, polypropylene, polycarbonate, styrene-acrylonitrile, polyethylene, or combinations thereof, it being understood that the inlet construction material may be different from the outlet construction material.

The filter 309 positioned in the flexible bag 300 can be secured to the interior surface of the sidewalls by any method known in the art. For example, sealing can be achieved by heat or impulse sealing to the interior surface of the first sidewall 305 at a first interface 313 located along a length of the filter bag and to the interior surface of the second sidewall 307 at a second interface 315 located along a length of the filter bag, wherein the first interface 313 is nearer to the inlet 301 than the second interface 315. The filter 309 can, in some embodiments, be a mesh filter constructed from a flexible material, e.g., polypropylene or polyester, and can have a mesh size of about 100 microns or less, such as from about 10 microns to about 80 microns, from about 20 microns to about 70 microns, from about 30 microns to about 60 microns, or from about 40 microns to about 50 microns, including all ranges and subranges therebetween.

The flexible flow-through apparatus design may have various advantages including, but not limited to, little or no molded parts, which may translate to a lower cost of manufacture. After use, the filter bag can contain the separated microbeads free or substantially free of media or cells, which can then be recovered by the user (e.g., by opening the bag). The filter bag may also be beneficial for preventing the retention of excess liquid by the filter.

Methods

The above-described flow-through apparatuses can be used to separate microbeads and/or cells from growth media. Methods for filtration include, in some embodiments, introducing a medium (e.g., including microbeads and/or cells) into a flow-through apparatus 100 or 200, wherein the medium enters the housing inlet and flows into the housing cavity through the first (and second) filter to produce a filtrate and the filtrate exits the housing cavity through the outlet. Other methods can include introducing a medium into a flow-through apparatus 300, wherein the medium enters the inlet and flows through the first filter to produce a filtrate and the filtrate exits through the outlet.

In the single-filter embodiment depicted in FIG. 3, the inlet 101 of the flow-through device 100 can be placed in fluid communication with the outlet of a flask or other container or apparatus (such as a Spinner flask or bioreactor) containing a medium to be filtered (e.g., including microbeads, cells, and growth media). The medium is introduced through the inlet 101 of the flow-through apparatus. Upon entering the flow-through apparatus, the medium enters the first filter 113, which is in fluid communication with the flow-through apparatus inlet 101. The first filter 113 has a mesh size of about 100 microns or less, which can capture the microbeads in the housing cavity while allowing passage of the cells and growth media through the first filter 113. The cells and growth media can thus pass through the first filter 113 as a filtrate and into the peripheral and basal gaps 131, 133, located between the outer surface of the first filter 113 and the inner surface of the filter housing 105, and exit the flow-through apparatus through the outlet 103. The cells may be separated from the filtrate in an additional downstream separation process. In some embodiments, a second flow-through apparatus similar to apparatus 100 can be placed downstream in the flow path, this apparatus being equipped with a filter having a smaller mesh size, e.g., a mesh size of about 5 microns or less, suitable for capturing and separating the cells from the medium. Of course, other conventional filtration methods for recovering cells from a medium can also be employed.

Recovery of the captured microbeads (or retentate) from flow-through apparatus 100 can include, for example, removing top 109 from the filter housing 105 (e.g., by unscrewing the top). The inner filter housing 111 may then be removed from the filter housing 105 and the locking or mating mechanism (e.g., “snap-fit”) between the first filter and the inner filter housing cap 123 can be disengaged, thereby allowing for the removal of the inner filter housing cap 123 and access to the first filter 113. Microbeads can be removed from the first filter 113, after which the first filter 113 may be replaced or washed and reused. The flow-through device may then be reassembled and reused for subsequent separations.

Filtration methods utilizing the flow-through apparatus 200 illustrated in FIG. 4, including a first filter and a second filter, can be substantially similar to the method described above with respect to the single-filter embodiment 100. In this embodiment, the first filter 213 is in fluid communication with the apparatus inlet 201 and the second filter 135. The cells and growth media pass through the first filter 213 and into the second filter 135, while the microbeads are trapped as a retentate in the first filter 213. The second filter 135 has a mesh size of about 5 microns or less, which can capture the cells and allow passage of the growth media through the second filter 135. The growth media passes through the second filter 135 and into the peripheral and basal gaps 131, 133 located between the outer surface of the second filter 135 and the inner surface of the filter housing 205 and then exits the flow-through device through the outlet 203.

The microbeads and cells can be recovered from the flow-through apparatus 200 in a substantially similar manner to the removal of the microbeads in the single-filter embodiment 100. After removal of the cap 209, the inner filter housing may then be removed from the filter housing 205 and the locking or mating mechanism (e.g., “snap-fit”) between the first filter and the inner filter housing cap 223 can be disengaged, thereby allowing for the removal of the inner filter housing cap 223 and access to the first filter 213. The first filter 213 (containing microbeads as a retentate) can then be removed from the inner filter housing thereby exposing the second filter 235 (containing cells as a retentate), which can also be removed. Microbeads and cells can be removed from the respective filters, and these filters can then be replaced or washed and reused. The flow-through device can be reassembled and reused for subsequent separations.

Filtration methods utilizing the flow-through apparatus 300 of FIG. 5 can include placing the inlet 301 in fluid communication with a container or apparatus containing a medium to be filtered (e.g., including microbeads, cells, and growth medium). The medium passes through the inlet 301 of the flow-through device and flows through the mesh filter 309 having a mesh size of about 100 microns or less, which is configured to capture the microbeads. A filtrate including cells and growth media (and substantially free of microbeads) exits the flow-through device through outlet 303. Cells in the filtrate may be separated in a downstream separation process. In some embodiments, a second flow-through apparatus similar to apparatus 300 can be placed downstream in the flow path, this apparatus being equipped with a filter having a smaller mesh size, e.g., a mesh size of about 5 microns or less, suitable for capturing and separating the cells from the medium. Of course, other conventional filtration methods for recovering cells from a medium can also be employed.

Recovery of microbeads from flow-through device 300 can be achieved by opening the flexible bag. In some embodiments, the flexible bag may be cut or torn open and the microbead retentate removed. The flow-through apparatus may then be discarded and replaced. According to additional embodiments, the bag 300 may have a seal that can be reversibly opened and closed such that microbeads can be removed and the bag washed and reused if desired. Similarly, if a second apparatus 300 is employed to separate cells from the media, this bag can be opened and optionally resealed to access the cell retentate within.

According to an aspect (1) of the present disclosure, a flow-through apparatus is provided. The flow-through apparatus comprises a housing having a fixed volume and comprising an inlet, an outlet, and a cavity; a first filter in fluid communication with the inlet and disposed within the cavity; and a seal attaching the first filter to the housing, wherein the first filter has a mesh size of about 100 microns or less and a first internal volume less than the fixed volume of the housing.

According to another aspect (2) of the present disclosure, the flow-through apparatus according to aspect (1) is provided wherein the first filter has a mesh size ranging from about 50 microns to about 80 microns.

According to another aspect (3) of the present disclosure, the flow-through apparatus according to any of aspects (1)-(2) is provided wherein the first filter has a cylindrical, conical, or frusto-conical shape.

According to another aspect (4) of the present disclosure, the flow-through apparatus according to any of aspects (1)-(3) is provided wherein the first internal volume ranges from about 10% to about 95% of the fixed volume of the housing.

According to another aspect (5) of the present disclosure, the flow-through apparatus according to any of aspects (1)-(4) is provided wherein the housing comprises a removable cap.

According to another aspect (6) of the present disclosure, the flow-through apparatus according to aspect (5) is provided wherein the seal attaching the first filter to the housing comprises an extending feature of the first filter secured between the removable cap and a lip of the housing.

According to another aspect (7) of the present disclosure, the flow-through apparatus according to any of aspects (1)-(6) is provided further comprising a second filter, wherein: the first filter is concentrically disposed within the second filter; and the second filter has a mesh size of about 5 microns or less and a second internal volume greater than the first internal volume of the first filter and less than the fixed volume of the housing.

According to another aspect (8) of the present disclosure, the flow-through apparatus according to aspect (7) is provided wherein the first and/or second filter has a cylindrical, conical, or frusto-conical shape.

According to another aspect (9) of the present disclosure, the flow-through apparatus according to any of aspects (7)-(8) is provided wherein the second internal volume ranges from about 10% to about 95% of the fixed volume of the housing.

According to another aspect (10) of the present disclosure, the flow-through apparatus according to any of aspects (7)-(9) is provided wherein the first internal volume ranges from about 10% to about 95% of the second internal volume.

According to another aspect (11) of the present disclosure, the flow-through apparatus according to any of aspects (1)-(10) is provided wherein the first filter is concentrically disposed within the cavity.

According to another aspect (12) of the present disclosure, a flow-through apparatus is provided. The flow through apparatus comprises a flexible bag comprising first and second sidewalls sealed along a periphery to define a cavity comprising an inlet and an outlet; and a filter disposed within the cavity and having a mesh size of about 100 microns or less, wherein the filter is attached to (a) an inside surface of the first sidewall at a first interface located along a length of the bag and (b) an inside surface of the second sidewall at a second interface located along the length of the bag, and wherein the length of the bag extends from the inlet to the outlet and the first interface is nearer to the inlet than the second interface.

According to another aspect (13) of the present disclosure, the flow-through apparatus according aspect (12) is provided wherein the filter has a mesh size ranging from about 50 microns to about 80 microns.

According to another aspect (14) of the present disclosure, the flow-through apparatus according to any of aspects (12)-(13) is provided wherein the first or second interface is located in a central region along the length of the bag.

According to another aspect (15) of the present disclosure, the flow-through apparatus according to any of aspects (12)-(14) is provided wherein the first interface is located proximate the inlet and/or the second interface is located proximate the outlet.

According to another aspect (16) of the present disclosure, a method for filtering a medium is provided. The method comprises introducing a medium into a flow-through apparatus comprising: a housing having a fixed volume and comprising an inlet, an outlet, and a cavity; a first filter in fluid communication with the inlet and disposed within the cavity, wherein the first filter has a mesh size of about 100 microns or less and a first internal volume less than the fixed volume of the housing; and a seal attaching the first filter to the housing, wherein the medium enters the inlet and flows into the cavity through the first filter to produce a filtrate and the filtrate exits the cavity through the outlet.

According to another aspect (17) of the present disclosure, the method according to aspect (16) is provided wherein the medium comprises microcarriers and optionally comprises cells, wherein the microcarriers are captured by the first filter, and wherein the filtrate is substantially free of microcarriers.

According to another aspect (18) of the present disclosure, the method according to any of aspects (16)-(17) is provided further comprising removing a retentate from the flow-through apparatus, wherein removing the retentate comprises: removing a removable cap from the housing; disengaging the seal attaching the first filter to the housing; removing the first filter from the housing; and removing the retentate from the first filter.

According to another aspect (19) of the present disclosure, the method according to any of aspects (16)-(18) is provided wherein the flow-through apparatus further comprises a second filter, and wherein: the first filter is concentrically disposed within the second filter; and the second filter has a mesh size of about 5 microns or less and a second internal volume greater than the first internal volume of the first filter and less than the fixed volume of the housing; and wherein the filtrate flows through the second filter to produce a second filtrate and the second filtrate exits the cavity through the outlet.

According to another aspect (20) of the present disclosure, the method according to aspect (19) is provided wherein the medium comprises microcarriers and cells, and wherein: the microcarriers are captured by the first filter, the filtrate is substantially free of microcarriers; the cells are captured by the second filter; and the second filtrate is substantially free of cells.

According to another aspect (21) of the present disclosure, the method according to any of aspects (19)-(20) is provided further comprising removing at least one retentate from the flow-through apparatus, wherein removing the retentate comprises: removing a removable cap from the housing; disengaging the seal attaching the first filter to the housing; removing the first filter from the housing; removing a first retentate from the first filter; removing the second filter from the housing; and removing a second retentate from the second filter.

According to another aspect (22) of the present disclosure, a method for filtering a medium is provided. The method comprises: introducing a medium into a flow-through apparatus comprising: a flexible bag comprising first and second sidewalls sealed along a periphery to define a cavity comprising an inlet and an outlet; and a filter disposed within the cavity and having a mesh size of about 100 microns or less, wherein the filter is attached to (a) an inside surface of the first sidewall at a first interface located along a length of the bag and (b) an inside surface of the second sidewall at a second interface located along the length of the bag, wherein the length of the bag extends from the inlet to the outlet and the first interface is nearer to the inlet than the second interface, and wherein the medium enters the inlet and flows through the first filter to produce a filtrate and the filtrate exits through the outlet.

According to another aspect (23) of the present disclosure, the method according to aspect (22) is provided wherein the medium comprises microcarriers and optionally comprises cells, wherein the microcarriers are captured by the filter, and wherein the filtrate is substantially free of microcarriers.

According to another aspect (24) of the present disclosure, the method according to any of aspects (12)-(23) is provided further comprising removing a retentate from the flow-through apparatus by unsealing or opening the flexible bag.

As used herein, the term “fluid communication” and variations thereof is intended to denote that a substance, e.g., medium including microbeads, cells, and growth media, can freely flow from one identified location to another. Fluid communication may be blocked and/or reestablished by closing and/or opening one or more components of the system, e.g., by closing a valve or blocking or clamping a conduit, or vice versa.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “a microcarrier” includes examples having two or more such “microcarriers” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 100 microns” and “a dimension less than about 100 microns” both include embodiments of “a dimension less than about 100 microns” as well as “a dimension less than 100 microns.”

The terms “substantial,” “substantially,” and variations thereof as used herein are intended to note that a described feature is equal or approximately equal to a value or description.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

It will be apparent to those skilled in the art that various modifications and variations can be made to the present disclosure without departing from the spirit and scope of the disclosure. Since modifications combinations, sub-combinations and variations of the disclosed embodiments incorporating the spirit and substance of the disclosure may occur to persons skilled in the art, the disclosure should be construed to include everything within the scope of the appended claims and their equivalents.

1. A flow-through apparatus comprising:
a housing having a fixed volume and comprising an inlet, an outlet, and a cavity;
a first filter in fluid communication with the inlet and disposed within the cavity; and
a seal attaching the first filter to the housing;
wherein the first filter has a mesh size of about 100 microns or less and a first internal volume less than the fixed volume of the housing.

2. The flow-through apparatus of claim 1, wherein the first filter has a mesh size ranging from about 50 microns to about 80 microns.

3. The flow-through apparatus of claim 1, wherein the first filter has a cylindrical, conical, or frusto-conical shape.

4. The flow-through apparatus of claim 1, wherein the first internal volume ranges from about 10% to about 95% of the fixed volume of the housing.

5. The flow-through apparatus of claim 1, wherein the housing comprises a removable cap.

6. The flow-through apparatus of claim 5, wherein the seal attaching the first filter to the housing comprises an extending feature of the first filter secured between the removable cap and a lip of the housing.

7. The flow-through apparatus of claim 1, further comprising a second filter, wherein:
the first filter is concentrically disposed within the second filter; and
the second filter has a mesh size of about 5 microns or less and a second internal volume greater than the first internal volume of the first filter and less than the fixed volume of the housing.

8. The flow-through apparatus of claim 7, wherein the first and/or second filter has a cylindrical, conical, or frusto-conical shape.

9. The flow-through apparatus of claim 7, wherein the second internal volume ranges from about 10% to about 95% of the fixed volume of the housing.

10. The flow-through apparatus of claim 7, wherein the first internal volume ranges from about 10% to about 95% of the second internal volume.

11. The flow-through apparatus of claim 1, wherein the first filter is concentrically disposed within the cavity.

12. A flow-through apparatus comprising:
a flexible bag comprising first and second sidewalls sealed along a periphery to define a cavity comprising an inlet and an outlet; and
a filter disposed within the cavity and having a mesh size of about 100 microns or less;
wherein the filter is attached to (a) an inside surface of the first sidewall at a first interface located along a length of the bag and (b) an inside surface of the second sidewall at a second interface located along the length of the bag; and
wherein the length of the bag extends from the inlet to the outlet and the first interface is nearer to the inlet than the second interface.

13. The apparatus of claim 12, wherein the filter has a mesh size ranging from about 50 microns to about 80 microns.

14. The apparatus of claim 12, wherein the first or second interface is located in a central region along the length of the bag.

15. The apparatus of claim 12, wherein the first interface is located proximate the inlet and/or the second interface is located proximate the outlet.

16. A method for filtering a medium, the method comprising:
introducing a medium into a flow-through apparatus comprising:
a housing having a fixed volume and comprising an inlet, an outlet, and a cavity;
a first filter in fluid communication with the inlet and disposed within the cavity, wherein the first filter has a mesh size of about 100 microns or less and a first internal volume less than the fixed volume of the housing; and
a seal attaching the first filter to the housing;
wherein the medium enters the inlet and flows into the cavity through the first filter to produce a filtrate and the filtrate exits the cavity through the outlet.

17. The method of claim 16, wherein the medium comprises microcarriers and optionally comprises cells, wherein the microcarriers are captured by the first filter, and wherein the filtrate is substantially free of microcarriers.

18. The method of claim 16, further comprising removing a retentate from the flow-through apparatus, wherein removing the retentate comprises:
removing a removable cap from the housing;
disengaging the seal attaching the first filter to the housing;
removing the first filter from the housing; and
removing the retentate from the first filter.

19. The method of claim 16, wherein the flow-through apparatus further comprises a second filter, and wherein:
the first filter is concentrically disposed within the second filter; and
the second filter has a mesh size of about 5 microns or less and a second internal volume greater than the first internal volume of the first filter and less than the fixed volume of the housing; and
wherein the filtrate flows through the second filter to produce a second filtrate and the second filtrate exits the cavity through the outlet.

20. The method of claim 19, wherein the medium comprises microcarriers and cells, and wherein:
the microcarriers are captured by the first filter,
the filtrate is substantially free of microcarriers;
the cells are captured by the second filter; and
the second filtrate is substantially free of cells.

21. The method of claim 19, further comprising removing at least one retentate from the flow-through apparatus, wherein removing the retentate comprises:
removing a removable cap from the housing;
disengaging the seal attaching the first filter to the housing;
removing the first filter from the housing;
removing a first retentate from the first filter;
removing the second filter from the housing; and
removing a second retentate from the second filter.

22. A method for filtering a medium, the method comprising:
introducing a medium into a flow-through apparatus comprising:
a flexible bag comprising first and second sidewalls sealed along a periphery to define a cavity comprising an inlet and an outlet; and
a filter disposed within the cavity and having a mesh size of about 100 microns or less;
wherein the filter is attached to (a) an inside surface of the first sidewall at a first interface located along a length of the bag and (b) an inside surface of the second sidewall at a second interface located along the length of the bag;
wherein the length of the bag extends from the inlet to the outlet and the first interface is nearer to the inlet than the second interface; and
wherein the medium enters the inlet and flows through the first filter to produce a filtrate and the filtrate exits through the outlet.

23. The method of claim 22, wherein the medium comprises microcarriers and optionally comprises cells, wherein the microcarriers are captured by the filter, and wherein the filtrate is substantially free of microcarriers.

24. The method of claim 22, further comprising removing a retentate from the flow-through apparatus by unsealing or opening the flexible bag.
